(19) United States
(12) Patent Application Publication
(10) Pub. No.: US 2022/0387746 A1
(43) Pub. Date: Dec. 8, 2022

(54) Method and apparatus for assessing an effect of a relaxation stimulus exposed to a human

(71) Applicant: MED STORM GO AS, Oslo (NO)

(72) Inventor: Hanne Storm, Oslo (NO)

(21) Appl. No.: 17/774,484

(22) PCT Filed: Oct. 28, 2020
(86) PCT No.: PCT/EP2020/080301

§ 371 Date: May 5, 2022

(30) Foreign Application Priority Data
Nov. 18, 2019 (NO) 20191366

(51) Int. Cl. A61M 21/02

(52) U.S. Cl. CPC A61M 21/02; A61M 2021/0027

(57) ABSTRACT

A method and an apparatus for assessing an effect of a relaxation stimulus exposed to a human have been disclosed. The method comprises the steps of providing a skin conductance signal measured at an area of the human's skin through a time interval; detecting peaks in the skin conductance signal through the time interval; determining a rate of the peaks through the time interval; and determining the effect of the relaxation stimulus based on the determined rate of peaks through the time interval.

### TECHNICAL FIELD

The invention relates in general to a method and an apparatus for assessing an effect of a relaxation stimulus exposed to a human.

### BACKGROUND OF THE INVENTION

During the recent years, several relaxation aids have emerged in the art. For instance, a large number of mobile device apps (e.g., smartphone apps) have become available, which claim to calm, relax or clear a human user's mind. Such relaxation aids include mobile device apps for guided meditation, yoga apps, hypnosis apps, calming music apps, etc.

It may be a difficult task to evaluate the effect of such relaxation aids. Hence, there is a need for a method and apparatus for assessing the effect of relaxation aids such as relaxation apps used on a mobile device, or more generally, there is a need for a method and an apparatus for assessing an effect of a relaxation stimulus exposed to a human.

### SUMMARY OF THE INVENTION

An object of the present invention is to provide a method and an apparatus for assessing an effect of a relaxation stimulus exposed to a human.

According to the invention, the above objects are achieved by a method and an apparatus as defined in the appended independent claims.

Further advantages and characteristics of the invention are indicated in the dependent claims.

### BRIEF DESCRIPTION OF THE DRAWINGS

The invention will be described by example with reference to the drawings, wherein

FIG. 1 is a schematic block diagram illustrating principles of an apparatus for assessing an effect of a relaxation stimulus exposed to a human,

FIG. 2 is a perspective view illustrating an apparatus for assessing an effect of a relaxation stimulus exposed to a human, during use, and

FIG. 3 is a flow chart illustrating a method for assessing an effect of a relaxation stimulus exposed to a human.

### DETAILED DESCRIPTION OF THE INVENTION

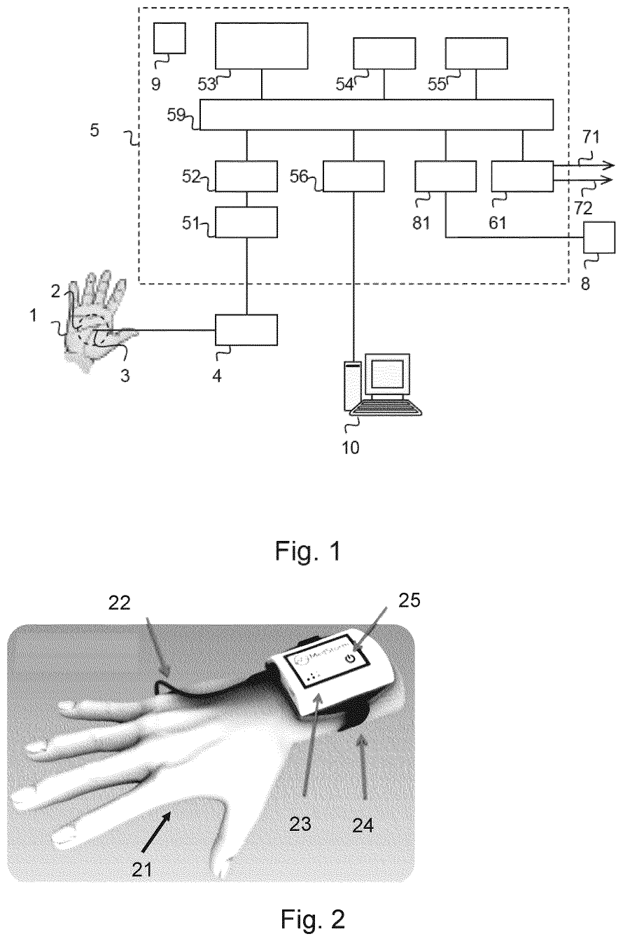

FIG. 1 is a schematic block diagram illustrating principles of an apparatus for assessing an effect of a relaxation stimulus exposed to a human.

On an area 2 of the skin on a body part 1 of the human, sensor means 3 are placed for measuring the skin's conductance. The body part 1 is preferably a hand or a foot, and the area 2 of the skin on the body part 1 is preferably the palmar side of the hand (in the palm of the hand) or the plantar side of the foot (under the sole of the foot). The sensor means 3 comprise contact electrodes where at least two electrodes are placed on the skin area 2. In a preferred embodiment the sensor means 3 consist of three electrodes: a signal electrode, a measuring electrode and a reference voltage electrode, which ensures a constant application of voltage over the stratum corneum (the surface layer of the skin) under the measuring electrode. The measuring electrode and the signal electrode are preferably placed on the skin area 2. The reference voltage electrode may also be placed on the skin area 2, but it is preferably placed in a nearby location, suitable for the measuring arrangement concerned.

In a preferred embodiment an alternating current is used for measuring the skin's conductance. The alternating current advantageously has a frequency in the range of up to 1000 Hz, corresponding to the area where the skin's conductance is approximately linear. A frequency should be selected which ensures that the measuring signal is influenced to the least possible extent by interference from, e.g., the mains frequency. In a preferred embodiment the frequency is 88 Hz. A signal generator, operating at the specified frequency, applies a signal current to the signal electrode.

In the case of alternating current the conductance is identical to the real part of the complex admittance, and therefore not necessarily identical with the inverse value of the resistance. An advantage of using alternating current instead of direct current in conductance measurement is that by this means one avoids the invidious effect on the measurements of the skin's electrical polarizing properties.

The resulting current through the measuring electrode is conveyed to a measurement converter 4. This comprises a current to voltage converter, which in a preferred embodiment is a trans resistance amplifier, but in its simplest form may be a resistance, which converts the current from the measuring electrode to a voltage.

The measurement converter further comprises a decomposition circuit, preferably in the form of a synchronous rectifier, which decomposes the complex admittance in a real part (the conductance) and an imaginary part (the susceptance). However, it is sufficient if the decomposition circuit only comprises means for deriving the conductance. The synchronous rectifier multiplies the measured voltage with the voltage from the signal generator. The two signals are in-phase. After multiplication, the result is according to the cosine (2u) equation, where the result is a DC component and one component at 2u frequency. In the preferred embodiment, this becomes 176 Hz. In the preferred embodiment, this synchronous rectifier is realized as an analog circuit with the required accuracy.

The measurement converter 4 may also comprise amplifier and filter circuits. In the preferred embodiment the measurement converter contains low-pass filters, both at the input and at the output. The object of the input low-pass filter is to attenuate high-frequency noise, for instance coming from other medical equipments, and also to serve as anti-aliasing filter to prevent high frequency components from being received by subsequent circuits for time discretization. The output low-pass filter shall attenuate the 2u components that result from the multiplication operation in the synchronous rectifier so that only the signal near DC is used for further processing.

By means of the choice of components and design details, moreover, the measurement converter is designed with a view to obtaining high sensitivity and a low noise level.

Although the measurement converter 4 has been illustrated to be external to the control unit 5 by example, it should be understood that the measurement converter may be included in the control unit 5.

The control unit 5 comprises a time discretization unit 51 for time discretization of the signal from the measurement converter. The time discretization takes place at a sampling rate, which may advantageously be in the order of 20 to 200 samplings per second. The control unit further comprises an analog-digital converter 52, which converts measurement data to digital form. The choice of circuits for time discretization and analog-digital conversion implies technical decisions suitable for a person skilled in the art. In the preferred embodiment, time discretization is done in an integrated circuit, which combines oversampling, filtering and discretization.

The control unit may advantageously comprise additional analog and possibly also digital inputs (not illustrated), in addition to the input from the measurement converter 4. In this case the control unit 5 can either be equipped with a plurality of analog-digital converters 52, or it can employ various multiplexing techniques well-known to those skilled in the art in order to increase the number of analog inputs. These additional analog inputs may, for example, be arranged for additional electrodermal measurements, or for other physiological measurements which may advantageously be performed simultaneously or parallel with the electrodermal measurement, such as temperature, pulse, ECG, respiratory measurements, oxygen saturation measurements in the blood, or EEG (bispectral index).

The control unit 5 also comprises a processing unit 53 for processing the digitized measurement data, storage means in the form of at least one store for storing data and programs, illustrated as a non-volatile memory 54 and a random access memory 55. The control unit 5 further comprises an interface circuit 61, which provides a first output signal 71 and optionally a further output signal 72. Optionally, the control unit 5 further comprises a further interface circuit 81, which is further connected to display unit 8. The control unit 5 may also optionally comprise a wireless communication adapter or a communication port 56 for digital communication with an external unit 10, such as a personal computer, a tablet, a mobile terminal or a smart telephone. Such communication is well-suited for loading or altering the program which is kept stored in the memory 54, 55 in the control unit, or for adding or altering other data which are kept stored in the memory 54, 55 in the control unit. Such communication is also well suited for read-out of data from the memory 54, 55 in the apparatus, thus enabling them to be transferred to the external unit 10 for further, subsequent analysis or storage.

In a preferred embodiment the non-volatile memory 54 comprises a read-only storage in the form of programmable ROM circuits, containing at least a program code and permanent data, and the random access memory 55 comprises a read and write storage in the form of RAM circuits, for storage of measurement data and other provisional data.

The control unit 5 also comprises an oscillator (not shown), which delivers a clock signal for controlling the processing unit 53. The processing unit 53 also contains timing means (not shown) in order to provide an expression of the current time, for use in the analysis of the measurements. Such timing means are well-known to those skilled in the art, and are often included in microcontrollers or processor systems which the skilled person will find suitable for use with the present invention.

The control unit 5 may be realized as a microprocessor-based unit with connected input, output, memory and other peripheral circuits, or it may be realized as a micro controller unit where some or all of the connected circuits are integrated. The time discretization unit 51 and/or analog-digital converter 52 may also be included in such a unit. The choice of a suitable form of control unit 5 involves decisions, which are suitable for a person skilled in the art.

An alternative solution is to realize the control unit 5 as a digital signal processor (DSP).

The control unit 5 is arranged to read time-discrete and quantized measurements for the skin conductance from the measurement converter 4, preferably by means of an executable program code, which is stored in the non-volatile memory 54 and which is executed by the processing unit 53. It is further arranged to enable measurements to be stored in the read and write memory 55. By means of the program code, the control unit 5 is further arranged to analyze the measurements in real time, i.e. simultaneously or parallel with the performance of the measurements. In this context, simultaneously or parallel should be understood to mean simultaneously or parallel for practical purposes, viewed in connection with the time constants which are in the nature of the measurements. This means that input, storage and analysis can be undertaken in separate time intervals, but in this case these time intervals, and the time between them, are so short that the individual actions appear to occur concurrently.

The control unit 5 is further arranged to detecting fluctuation peaks in the time-discrete, quantized measuring signal, by means of a program code portion which is stored in the non-volatile memory 54 and which is executed by the processing unit 53.

The control unit 5 is further arranged to determining the rate of the fluctuation peaks in the time-discrete, quantized measuring signal through a time interval, by means of a program code portion which is stored in the non-volatile memory 54 and which is executed by the processing unit 53.

The control unit 5 is further arranged to determining the effect of the relaxation stimulus based on the determined rate of peaks through the time interval, by means of a program code portion which is stored in the non-volatile memory 54 and which is executed by the processing unit 53.

The control unit 5 is further arranged to provide a first output signal 71 of an interface circuit 61, the output signal including output data representative of the determined effect of the relaxation stimulus, by means of a program code portion which is stored in the non-volatile memory 54 and which is executed by the processing unit 53.

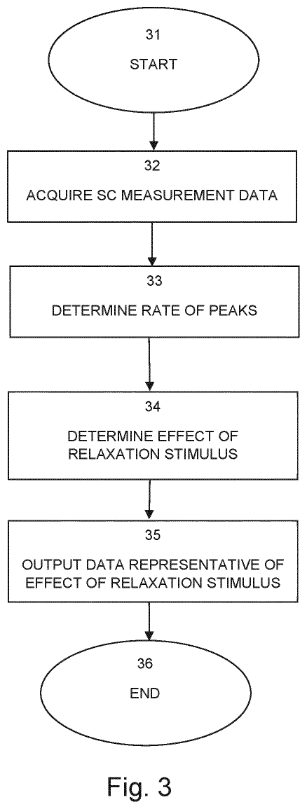

The control unit 5 may further be arranged to perform various steps and combinations of steps of the method as disclosed herein, in particular with reference to FIG. 3 below, by means of a program code portion which is stored in the non-volatile memory 54 and which is executed by the processing unit 53.

The processing unit 53, the memories 54, 55, the analog/digital converter 52, the communication port 56, the interface circuit 81 and the interface circuit 61 are all connected to a bus unit 59. The detailed construction of such bus architecture for the design of a microprocessor-based instrument is regarded as well-known for a person skilled in the art.

The interface circuit 61 is a digital port circuit, which derives at least a first digital output signal 71, from the processing unit 53 via the bus unit 59 when the interface circuit 61 is addressed by the program code executed by the processing unit 53.

The first digital output signal 71 indicates output data representative of the determined effect of the relaxation stimulus.

Additional output signals, schematically illustrated as a second output signal 72, may also be provided by the interface circuit 61.

In a preferred embodiment the display means 8 consist of a screen for graphic visualization of the conductance signal, and a digital display for displaying the frequency and amplitude of the measured signal fluctuations. The display units are preferably of a type whose power consumption is low, such as an LCD screen and LCD display. The display means may be separate or integrated in one and the same unit.

The apparatus further comprises a power supply unit 9 for supplying operating power to the various parts of the apparatus. The power supply may include a battery, preferably a rechargeable battery, or alternatively a connection to an external power supply such as a mains supply.

FIG. 2 is a perspective view illustrating an apparatus for assessing an effect of a relaxation stimulus exposed to a human, during use.

Electrodes, typically three electrodes as described above with reference to the disclosure of FIG. 1, are placed on the palmar side of a hand 21 of a human, hence the electrodes are not visible on FIG. 2. The electrodes are interconnected by a cable 22 to an apparatus 23 for assessing an effect of a relaxation stimulus exposed to the human. The apparatus 23 includes a housing which encapsulates electronics that embodies the measurement converter 4 and the control unit 5 as described above with reference to FIG. 2. The housing of the apparatus is strapped to the wrist of the human by means of a suitable wrist strap 24, preferably made of an elastic material. The apparatus 23 includes a simple operational interface including an operational element 25 that may be a touch button for switching the apparatus on and off. The apparatus 23 also includes an indicator, that may be an optical or audible indicator, or both, that provides an optical or audible indication corresponding to the first output signal 71, indicating data representative of the determined effect of the relaxation stimulus. Further operation of the apparatus may be provided by means of an external terminal, such as a wireless terminal or a smartphone, corresponding to the external unit 10 and enabled to communicate with the control unit 5 in the apparatus 23 as described above with reference to FIG. 1. Particularly, in the case that the method includes an introductory step of exposing the human to the relaxation stimulus, the control unit may be configured to initiate the execution of an application program on the external unit 10, the application program may, e.g., be a meditation app, a yoga app, a hypnosis app and a relaxational music app, and the external unit 10 is arranged to expose the relaxation stimulus, in particular audio data provided by the application program, to the human's hearing and optionally visual senses. This may e.g. be achieved by means of headphones used by the human and connected by wire or wirelessly to the external unit 10, or by speakers exposed to the human ears and connected by wire or wirelessly to the external unit 10.

FIG. 3 is a flow chart illustrating a method for assessing an effect of a relaxation stimulus exposed to a human.

The method starts at reference 31.

In measurement step 32, a skin conductance signal measured at an area of the human's skin through a time interval is provided. To this end, skin conductance signal or EDR (electrodermal response) signal is measured by means of sensor means 3 such as contact electrodes, arranged on a body part 1 of the human, preferably the palmar side of a hand, as already has been explained with reference to FIGS. 1 and 2 above.

The skin conductance, preferably in microsiemens (uS), is time-quantized and converted to digital form, for instance by the use of equipment described with reference to FIG. 1 above. A time-series of a certain duration, typically between 1 and 10 minutes, and more preferably between 2 and 8 minutes, still more preferably between 4 and 6 minutes, containing skin conductance data, is acquired during step 32.

In the subsequent peaks rate determining step 33, peaks in the skin conductance signal are detected through the time interval, and a rate of the peaks through the time interval is determined.

In the peak detecting substep of step 33, a test may be performed in order to detect the existence of valid peaks in the time-series of acquired skin conductance signal. If one or more peaks are detected, the rate of peaks is subsequently determined. If no peak is detected, the rate of peaks is considered to be zero.

The existence of a valid peak may be established if the derivative of the signal changes sign through a small period in the interval. The derivative of the signal may be calculated as the difference between two subsequent sample values. In addition, it is possible to use a simple digital filter that needs to see two or more subsequent sign changes before the sign change is accepted.

In the test step of establishing a valid peak it may be necessary to establish additional criteria for when a peak should be considered as valid. In their simplest form such criteria may be based on the fact that the signal amplitude has to exceed an absolute limit in order to be able to be considered a valid fluctuation. A recommended, such reference value for the conductance is between 0.01 μS and 0.02 μS, preferably 0.015 μS.

Alternatively or in addition, it may be advantageous to base the criteria on the fact that the signal actually has formed a peak that has lasted a certain time. The criteria may also be based on the fact that the increase in the skin conductance signal value as a function of time must remain below a certain limit, typically 20 μS/s, if the maximum value is to be considered valid.

Another possible condition for establishing a valid peak is that the absolute value of the change in the conductance signal from a local peak to the following local valley exceeds a predetermined value, such as between 0.01 μS and 0.02 μS, preferably 0.015 μS.

Also, a maximum value appearing at the border of the interval, i.e. the starting point or ending point of the interval should preferably not be regarded as a valid peak.

The object is thereby achieved that artifacts, which can occur in error situations such as, e.g., electrodes working loose from the skin, or other sources of noise or disturbances, does not lead to the erroneously detection of peaks.

In the effect determining step 34, the effect of the relaxation stimulus based on the determined rate of peaks through the time interval is determined.

Advantageously, a lower determined rate of peaks through the interval results in the determination of an improved effect of the relaxation stimulus, i.e., a successful relaxation in the human.

Although not illustrated in FIG. 3, the method may advantageously further comprise determining a value representative of the derivative of the skin conductance signal through the time interval. In this case, the effect of the relaxation stimulus may advantageously further be based on the determined value representative of the derivative of the skin conductance signal through the time interval.

In particular, a negative derivative of the skin conductance signal through a substantial part of the time interval, or through the entire time interval, is an indicator of a successful relaxation in the human.

Advantageously, the step of determining the effect of the relaxation stimulus based on the determined rate of peaks may comprise mapping the determined rate of peaks to a relaxation success index.

For instance, the mapping of the determined rate of peaks to the relaxation success index may be performed by entries in the following table:

| Determined rate of peaks | Relaxation |
|---|---|
| [No. of peaks/minute], | success |
| interval | index |
| 0.00 | 100% |
| 0.08 | 90% |
| 0.10 | 70% |
| 0.15 | 50% |
| 0.20 | 20% |
| 0.33 or more | 0% |

For instance, the mapping of the determined derivative of the mean skin conductance level to the relaxation success index may be performed by entries in the following table:

| Determined derivate of | |
|---|---|
| the mean skin | Relaxation |
| conductance level/ | success |
| minute, interval | index |
| −0.10 or more | 100% |
| −0.08 | 90% |
| −0.05 | 70% |
| −0.02 | 50% |
| −0.01 | 20% |
| −0.00 or positive | 0% |

For instance, the mapping of the determined derivative of the mean skin conductance level and the decrease in the peaks per sec to the relaxation success index may be performed by entries in the following table:

| Determined derivate of | | |
|---|---|---|
| the mean skin | Determined rate of peaks | Relaxation |
| conductance level/ | [No. of peaks/minute], | success |
| minute, interval | interval | index |
| −0.10 or more | 0.00 | 100% |
| −0.08 | 0.08 | 90% |
| −0.05 | 0.10 | 70% |
| −0.02 | 0.15 | 50% |
| −0.01 | 0.20 | 20% |
| −0.00 or positive | 0.33 or more | 0% |

Advantageously, the effect determining step 34 is followed by the data output step 35, in which output data representative of the determined effect of the relaxation stimulus is provided. For instance, the output data may be provided as the output signal 71 illustrated in FIG. 1 above.

The output data may, e.g., be provided as a number or a graphic representation, for instance by the use of colour codes or other graphical elements representing the relaxation success index.

Subsequent to the data output step 35, the process may end at the terminating step 36, or alternatively, it may be repeated by continuing at the measurement step 32.

Although not illustrated in FIG. 3, an introductory step of exposing the human to the relaxation stimulus may be included in the method. This introductory step may include initiating the execution of an application program on the external unit 10, the application program may, e.g., be a meditation app, a yoga app, a hypnosis app or a relaxational music app. The external unit 10 may then be arranged to expose the relaxation stimulus, in particular audio data provided by the application program, to the human's hearing sense. This may e.g. be achieved by means of headphones used by the human and connected to the external unit 10, or by speakers exposed to the human ears and connected to the external unit 10.

As an alternative or addition to the meditation app, yoga app, hypnosis app, a relaxational music app, etc., the introductory step of exposing the human to the relaxation stimulus may include a real time experience with a person in site talking and/or providing visual stimuli distracting the human into a relaxed situation.

The process may at any time be interrupted or terminated by an operating device (not shown) or by a command input from the communication port 56.

The disclosed method and apparatus results in a reliable establishment of assessing an effect of a relaxation stimulus exposed to a human.

1. Method for assessing an effect of a relaxation stimulus exposed to a human, the method comprising the steps of:
providing a skin conductance signal measured at an area of the human's skin, preferably on a palmar side of a hand or a plantar side of a foot, through a time interval;
detecting peaks in the skin conductance signal through the time interval;
determining a rate of the peaks through the time interval;
determining a value representative of the derivative of the skin conductance signal through the time interval
determining the effect of the relaxation stimulus based on the determined rate of peaks through the time interval and the determined value representative of the derivative of the skin conductance signal through the time interval.

2. Method according to claim 1, wherein the step of determining the effect of the relaxation stimulus based on the determined rate of peaks and the derivative of the skin conductance comprises mapping the determined derivative of the mean skin conductance level and the determined rate of peaks to a relaxation success index according to:

| Determined derivate of | | |
|---|---|---|
| the mean skin | Determined rate of | Relaxation |
| conductance level/ | peaks [No. of | success |
| minute, interval | peaks/minute], interval | index |
| −0.10 or more | 0.00 | 100% |
| −0.08 | 0.08 | 90% |
| −0.05 | 0.10 | 70% |
| −0.02 | 0.15 | 50% |
| −0.01 | 0.20 | 20% |
| −0.00 or positive | 0.33 or more | 0% |

3. Method according to claim 1, wherein the time interval is in the range 1 to 10 minutes, or wherein the time interval is in the range 2 to 8 minutes, or wherein the time interval is in the range 4 to 6 minutes.

4. Method according to claim 1, wherein said step of detecting peaks in the skin conductance signal through said time interval comprises establishing the existence of a valid peak if the derivative of the skin conductance signal changes sign through a small period in the interval.

5. Method according to claim 1, further comprising providing output data representative of the determined effect of the relaxation stimulus.

6. Method according to claim 1, wherein the relaxation stimulus exposed to the human includes audio data exposed to the human's hearing sense and optionally visual data exposed to the human's visual sense.

7. Method according to claim 1, wherein the relaxation stimulus is provided by a wireless terminal which executes an application program, the application program being selected from a meditation app, a yoga app, a hypnosis app and a relaxational music app.

8. Method according to claim 1, further comprising the introductory step of exposing the human to the relaxation stimulus.

9. Apparatus for assessing an effect of a relaxation stimulus exposed to a human, comprising
measurement equipment for providing a skin conductance signal measured at an area of the patient's skin, and
a control unit, configured to perform a method as set forth in one of the claims 1-8.
